(19) United States
(12) Patent Application Publication
(10) Pub. No.: US 2023/0203493 A1
(43) Pub. Date: Jun. 29, 2023

(54) Biomarkers Related to Oral Squamous Cell Carcinoma and Methods of Diagnosis and Treatment Thereof

(71) Applicant: QINGDAO MEDINTELL BIOMEDICAL CO., LTD, Qingdao, Shandong (CN)

(72) Inventors: Chenggang YANG, Qingdao, Shandong (CN); Feng XIAO, Qingdao, Shandong (CN)

(21) Appl. No.: 18/000,197

(22) PCT Filed: May 30, 2021
(86) PCT No.: PCT/CN2021/097061

§ 371 Date: Nov. 29, 2022

(30) Foreign Application Priority Data
May 31, 2020 (CN) 202010481200.9
May 31, 2020 (CN) 202010481203.2
May 31, 2020 (CN) 202010481204.7
May 31, 2020 (CN) 202010481206.6
May 31, 2020 (CN) 202010481207.0
May 31, 2020 (CN) 202010481208.5
May 31, 2020 (CN) 202010481211.7

(51) Int. Cl. C12N 15/113; C12Q 1/6886; A61K 49/00; A61P 35/00

(52) U.S. Cl. CPC C12N 15/1135; C12Q 1/6886; A61K 49/0004; A61P 35/00; C12Q 2600/136; C12Q 2600/158; C12N 2310/14

(57) ABSTRACT

Biomarkers are related to oral squamous cell carcinoma and can be used in methods of diagnosis and treatment of oral squamous cell carcinoma The biomarkers are lncRNA RP11-875O11.3, LINC01679, AP000695.4, RP11-339B21.10, RP11-426C22.4, RP11-426C22.5 and/or AP000695.6 genes. The biomarkers are used in the preparation of products for diagnosing oral squamous cell carcinoma and products for diagnosing oral squamous cell carcinoma. The biomarkers are also used in the preparation of a pharmaceutical composition for treating oral squamous cell carcinoma and a pharmaceutical composition for treating oral squamous cell carcinoma.

### CROSS-REFERENCE TO RELATED APPLICATIONS

This application is a U.S. national stage entry of PCT International Application No. PCT/CN2021/097061, filed on May 30, 2021, which claims priority to Chinese Patent Applications, filed to China National Intellectual Property Administration on May 31, 2020, No. 202010481211.7, entitled “Use of lncRNA biomarker in the diagnosis and treatment of oral squamous cell carcinoma”; No. 202010481200.9, entitled “Use of reagents for detecting and targeting biomarkers in oral squamous cell carcinoma”; No. 202010481208.5, entitled “New use of biomarkers”; No. 202010481207.0, entitled “Use of biomarker-based diagnosis and treatment of cancer”; No. 202010481206.6, entitled “Biomarkers for diagnosis and treatment of oral squamous cell carcinoma”; No. 202010481204.7, entitled “Relevant biomarkers and applications for diagnosis and treatment of oral squamous cell carcinoma”; No. 202010481203.2, entitled “Biomarkers for Predicting Oral Squamous Cell Carcinoma and Use thereof in treatment”, the content of each is incorporated herein by reference in its entirety.

### INCORPORATION OF SEQUENCE LISTING

This application contains a sequence listing submitted in Computer Readable Form (CRF). The CFR file containing the sequence listing entitled “PA630-0007_ST25.txt”, which was created on Nov. 29, 2022, and is 6,108 bytes in size. The information in the sequence listing is incorporated herein by reference in its entirety.

### TECHNICAL FIELD

The present invention belongs to the field of biomedicine and relates to a biomarker related to oral squamous cell carcinoma and methods of diagnosis and treatment thereof.

### BACKGROUND ART

Oral squamous cell carcinoma (Oral squamous cell carcinoma, OSCC) is an epithelial-derived malignant tumor that is prone to metastasis and is the eleventh most common cancer worldwide (Hussein A A, Helder M N, de Visscher J G, et al. Global incidence of oral and oropharynx cancer in patients younger than 45 years versus older patients: A systematic review[J]. EurJCancer 2017; 82:115-127.). About 600,000 new cases occur each year, making it the 15^{th} most common cause of cancer deaths in the world (Candia J, Fernandez A, Somarriva C, et al. Deaths due to oral cancer in Chile in the period 2002-2012[J]. Rev Med Chil. 2018; 146(4):487-493.). In recent years, the global incidence of oral squamous cell carcinoma has increased significantly, and the age of onset has gradually become younger, but the causes of the change in the incidence are not clear. Especially in developing countries, the rate of incidence growth is particularly prominent (Wang F, Zhang H, Wen J, et al. Nomograms forecasting long-term overall and cancer-specific survival of patients with oral squamous cell carcinoma[J]. Cancer Med. 2018; 7(4):943-952.).

With the improvement of imaging, surgery, radiotherapy and traditional treatment, the current treatment of OSCC is mainly surgical resection, chemotherapy, and radiotherapy or a combination of these three methods (Kim S M, Jeong D, Min K K, et al. Two different protein expression profiles of oral squamous cell carcinoma analyzed by immunoprecipitation high-performance liquid Chromatography[J]. World Journal of Surgical Oncology 0.2017; 15(1):151.). Despite the continuous improvement in treatment methods, oral surgery has been severely limited in scope due to close contact with vital tissues and organs. In addition, neck and facial tissues are rich in blood vessels and nerves, with a high incidence of cervical lymph node metastasis and invasion and poor prognosis, and the 5-year survival rate has not increased significantly (about 50%-60%) in recent years. Patients with advanced tumors or tumor recurrence have a lower 5-year survival rate (Radhika T, Jeddy N, et al. Salivary biomarkers in oral squamous cell carcinoma—An insight [J]. Journal of Oral Biology&Craniofacial Research 2016, 6(Suppl 1): S51-54.). Studies have shown that some patients with advanced OSCC who had surgical resection at the beginning are also alive for less than 30 months (Felice F D, Polimeni A, et al. Radiotherapy Controversies and Prospective in Head and Neck Cancer: A Literature-Based Critical Review[J]. Neoplasia 2018; 20(3):227-232.). In addition, the 5-year survival rate of patients is also correlated with the location and stage of the tumor, patient age, and whether there is an underlying disease. Therefore, for OSCC, finding tumor markers with a molecular diagnosis, prognosis prediction, and targeted therapy is of great significance for the treatment of tumors, and is the future direction of development. An in-depth understanding of the occurrence, development, invasion, and metastasis mechanism of OSCC, revealing the pro-oncogene and anti-oncogene of OSCC is conducive to improving and supplementing the treatment of oral squamous cell carcinoma, which has important clinical significance.

### SUMMARY OF THE INVENTION

To make up for the deficiencies of the prior art, the present invention studies genes that are differentially expressed in oral squamous cell carcinoma and explores the effect of differentially expressed genes on cancer cells through further cell experiments, thereby providing detection and targeting sites for the diagnosis and treatment of oral squamous cell carcinoma and providing a theory for revealing the pathogenesis of oral squamous cell carcinoma.

The present invention adopts the following technical solution:

One aspect of the present invention provides a biomarker for diagnosing oral squamous cell carcinoma selected from one or more of RP11-875O11.3, LINC01679, AP000695.4, RP11-339B21.10, RP11-426C22.4, RP11-426C22.5, AP000695.6.

Further, the biomarker is significantly up-regulated in oral squamous cell carcinoma compared to normal (para-cancerous) samples.

A second aspect of the present invention provides use of the biomarker according to the first aspect of the present invention and/or expression product thereof or use of reagents for specifically detecting the biomarker according to the first aspect of the present invention and/or expression product thereof in the preparation of products for the diagnosis of oral squamous cell carcinoma. Further, the reagent is selected from: primers that specifically amplify RP11-875O11.3, LINC01679, AP000695.4, RP11-339B21.10, RP11-426C22.4, RP11-426C22.5 and/or AP000695.6 genes; or probes that specifically recognize RP11-875O11.3, LINC01679, AP000695.4, RP11-339B21.10, RP11-426C22.4, RP11-426C22.5 and/or AP000695.6 genes.

Further, the primer sequences for specifically amplifying RP11-875O11.3, LINC01679, AP000695.4, RP11-339B21.10, RP11-426C22.4, RP11-426C22.5 and/or AP000695.6 genes are shown in SEQ ID Nos: 1-14, respectively.

A third aspect of the invention provides a product for diagnosing oral squamous cell carcinoma comprising reagents for detecting the biomarker according to the first aspect of the invention. Further, the product comprises a chip, a kit, or a test strip. Wherein the chip comprises a solid support and an oligonucleotide probe immobilized on the solid support, the oligonucleotide probe comprises an oligonucleotide probe for a biomarker for detecting the expression level of the biomarker; the kit comprises a primer, a probe, or a chip for detecting the expression level of the biomarker.

Further, the kit further comprises instructions for use or a label, a positive control, a negative control, a buffer, an adjuvant, or a solvent; the instructions or label indicates that the kit is used to detect oral squamous cell carcinoma.

Further, the reagents comprise reagents for detecting the biomarker of the present invention by reverse transcription PCR, real-time quantitative PCR, in situ hybridization, or gene chip.

Further, the reagents for detecting the biomarker of the present invention by reverse transcription PCR comprise at least one pair of primers for specifically amplifying the biomarker; the reagents for detecting the biomarker of the present invention by real-time quantitative PCR comprise at least one pair of primers for specifically amplifying the biomarker; the reagents for detecting the biomarker of the present invention by in situ hybridization comprise probes that hybridize to nucleic acid sequences of the biomarker; the reagents for detecting the biomarker of the present invention by gene chip comprise probes that hybridize to nucleic acid sequences of the biomarkers.

A fourth aspect of the invention provides the use of the biomarker according to the first aspect of the invention in the preparation of a pharmaceutical composition for treating oral squamous cell carcinoma.

Further, the pharmaceutical composition comprises an inhibitor of the functional expression of the biomarker.

Further, the inhibitor reduces the expression level of one or more biomarkers.

Further, the inhibitor is selected from the group consisting of gapmer, interference RNA, CRISPR, TALEN, or zinc finger nucleases.

Further, the inhibitor is selected from interference RNA.

In a specific embodiment of the present invention, the interference RNA is siRNA, and the sequence is as follows:

the siRNA sequence of RP11-875O11.3 is shown in SEQ ID NO: 17 and SEQ ID NO: 18;

the siRNA sequence of LINC01679 is shown in SEQ ID NO: 19 and SEQ ID NO: 20;

the siRNA sequence of AP000695.4 is shown in SEQ ID NO: 21 and SEQ ID NO: 22;

the siRNA sequence of RP11-339B21.10 is shown in SEQ ID NO: 23 and SEQ ID NO: 24;

the siRNA sequence of RP11-426C22.4 is shown in SEQ ID NO: 25 and SEQ ID NO: 26;

the siRNA sequence of RP11-426C22.5 is shown in SEQ ID NO: 27 and SEQ ID NO: 28;

the siRNA sequence of AP000695.6 is shown in SEQ ID NO: 29 and SEQ ID NO: 30.

A fifth aspect of the invention provides a pharmaceutical composition comprising an inhibitor of functional expression of the biomarker according to the first aspect of the invention.

Further, the inhibitor reduces the expression level of one or more biomarkers.

Further, the inhibitor is selected from the group consisting of gapmer, interference RNA, CRISPR, TALEN, or zinc finger nucleases.

Further, the inhibitor is selected from interference RNA.

In a specific embodiment of the invention, the interference RNA is siRNA, the siRNA has the sequence SEQ ID Nos: 17-30 as described above.

Further, the pharmaceutical composition further comprises a pharmaceutically acceptable carrier.

A sixth aspect of the invention provides the use of the biomarker according to the first aspect of the invention in the screening of a candidate drug for treating oral squamous cell carcinoma.

Further, the step of screening a candidate drug is as follows:

(1) treating a system expressing or containing the biomarker according to the first aspect of the invention with a substance to be screened; and

(2) detecting the expression level of the biomarker in the system;

if the substance to be screened reduces the expression level of the biomarker, it indicates that the substance to be screened is a candidate drug for preventing or treating oral squamous cell carcinoma.

Further, such candidate substance includes, but is not limited to: an interfering molecule, a nucleic acid inhibitor, a binding molecule, a small molecule compound, etc. targeting the biomarker or an upstream or downstream gene thereof.

A seventh aspect of the invention provides a method of screening a candidate drug for preventing or treating oral squamous cell carcinoma, the method comprising:

(1) treating a system expressing or containing the biomarker according to the first aspect of the invention with a substance to be screened; and

(2) detecting the expression level of the biomarker in the system;

if the substance to be screened reduces the expression level of the biomarker, it indicates that the substance to be screened is a candidate drug for preventing or treating oral squamous cell carcinoma.

An eighth aspect of the invention provides a method of inhibiting the proliferation of a tumor cell by introducing into the tumor cell an inhibitor of the biomarker according to the first aspect of the invention.

Further, the inhibitor comprises a siRNA, an shRNA, an antisense oligonucleotide, or a loss-of-function gene for the biomarker.

A ninth aspect of the invention provides a method of diagnosing oral squamous cell carcinoma comprising: detecting the expression level of the biomarker according to the first aspect of the invention in a sample from a subject.

If the expression of at least one of RP11-875O11.3, LINC01679, AP000695.4, RP11-339B21.10, RP11-426C22.4, RP11-426C22.5 and AP000695.6 in the sample of the subject is significantly increased as compared with that in the normal human, the subject is diagnosed as an oral squamous cell carcinoma patient.

Further, the method comprises:

(1) collecting subject samples;

(2) extracting RNA from a sample from a subject and detecting the expression level of the biomarker according to the first aspect of the invention;

(3) if the expression of at least one of RP11-875O11.3, LINC01679, AP000695.4, RP11-339B21.10, RP11-426C22.4, RP11-426C22.5 and AP000695.6 in the sample of the subject is significantly increased as compared with that in the normal human, the subject is diagnosed as an oral squamous cell carcinoma patient.

A tenth aspect of the invention provides a method of preventing or treating oral squamous cell carcinoma comprising: administering to the subject a pharmaceutically effective amount of an inhibitor of the biomarker of the first aspect of the invention.

Further, the inhibitor reduces the expression level of one or more biomarkers.

Further, the inhibitor is selected from the group consisting of gapmer, interference RNA, CRISPR, TALEN, or zinc finger nucleases.

Further, the inhibitor is selected from interference RNA.

Further, the sequences of interference RNA are selected from the group consisting of SEQ ID Nos: 17-30.

Another aspect of the invention provides a method of inhibiting tumor cell proliferation by introducing a down-regulator of the RP11-875O11.3 gene into a tumor cell in vitro.

Further, the down-regulator comprises a siRNA, an shRNA, an antisense oligonucleotide or a loss-of-function gene targeting RP11-875O11.3 gene.

### DETAILED DESCRIPTION OF THE INVENTION

After an in-depth study, the present invention has found for the first time that the expression of RP11-875O11.3, LINC01679, AP000695.4, RP11-339B21.10, RP11-426C22.4, RP11-426C22.5 and AP000695.6 genes in oral squamous cell carcinoma tissues is significantly higher than that in normal mucosa tissues, and has experimentally demonstrated that RP11-875O11.3, LINC01679, AP000695.4, RP11-339B21.10, RP11-426C22.4, RP11-426C22.5, and AP000695.6 also showed high expression in oral squamous carcinoma cells, and down-regulating the expression level of RP11-875O11.3, LINC01679, AP000695.4, RP11-339B21.10, RP11-426C22.4, RP11-426C22.5, and AP000695.6 could inhibit the proliferation and invasion of oral squamous carcinoma cells, suggesting RP11-875O11.3, LINC01679, AP000695.4, RP11-339B21.10, RP11-426C22.4, RP11-426C22.5, and AP000695.6 can be used as a target for diagnosis and treatment in clinic.

The lncRNA of the present invention comprises wild-type, mutant, or fragments thereof, as long as it can be aligned to the gene during sequence alignment. Presently disclosed RP11-875O11.3 exists as two transcripts with sequences shown in ENST00000520840.1 and ENST00000523806.1, respectively. In a specific embodiment of the present invention, the sequence of RP11-875O11.3 is shown in ENST00000520840.1. Presently disclosed LINC01679 exists as one transcript with sequences as shown in NR_131902.1. Presently disclosed AP000695.4 exists as two transcripts with sequences shown as ENST00000428667.1 and ENST00000454980.1, respectively. In a specific embodiment of the present invention, the sequence of AP000695.4 is shown in ENST00000428667.1. Presently disclosed RP11-339B21.10 exists as one transcript with sequences shown in ENST00000610052.1. Presently disclosed RP11-426C22.4 exists as one transcript with sequences shown in ENST00000566070.1. Presently disclosed RP11-426C22.5 exists as two transcripts with sequences shown in ENST00000562902.1 and ENST00000563477.1, respectively. In a specific embodiment of the present invention, the sequence of RP11-426C22.5 is shown in ENST00000562902.1. Presently disclosed AP000695.6 exists as one transcript with sequences shown in ENST00000429588.1.

As used herein, “marker” and “biomarker” may be used in combination to refer to a target molecule that is indicative of normal or abnormal progression in an individual or indicative of or expression of a disease or other condition in an individual. In more detail, a “marker” or “biomarker” is normal or abnormal, and if abnormal, an anatomical, physiological, biochemical, or molecular parameter associated with the presence of a particular physiological state or progression that is chronic or acute. Biomarkers can be detected and measured by a variety of methods including laboratory detection and medical imaging.

As used herein, “biomarker value”, “value”, “biomarker level”, and “level” are determined using any analytical method for detecting a biomarker from a biological sample, and which, in the above biological samples, are used interchangeably to refer to indicating or used as a biomarker, corresponding to the existence or non-existence of biomarkers, absolute quantity or concentration, relative quantity or concentration, titrate, level, expression level, the ratio of measured level, etc. “Diagnosing”, “diagnosed”, “diagnosis”, and variations of these terms refer to the discovery, judgment, or recognition of a health state or condition of an individual based on one or more signs, symptoms, data, or other information related to the individual. The health status of an individual may be diagnosed as healthy/normal (i.e., the absence of a disease or condition) or may be diagnosed as unhealthy/abnormal (i.e., the presence of an assessment of a disease or condition, or characteristic). The above terms “diagnosing”, “diagnosed”, “diagnosis”, and the like comprise the early detection of a disease associated with a particular disease or condition; the nature or classification of the disease; the discovery of progression, cure, or recurrence of a disease; the discovery of a response to disease following treating or treatment of an individual. The diagnosis of oral squamous cell carcinoma comprises the distinction between individuals who do not have cancer and those who do.

When a biomarker is one that indicates abnormal progression or disease or other state or a marker thereof in an individual, the biomarker typically indicates the absence of normal progression or disease or other states in the individual, or exhibits one of over-expression or under-expression compared to the expression level or value of the biomarker as the marker. “Upregulation”, “up-regulated”, “overexpression”, and variations of the expression are used interchangeably to refer to a biomarker value or level in a biological sample with a higher value or level (or range of values or levels) than those typically detected from biological sample similar to healthy or normal individuals. Multiple of the above terms may also refer to a biomarker value or level in a biological sample that is higher than the value or level (or range of values or levels) of a biomarker that may be detected in different steps of a particular disease.

“Downregulation”, “underexpression”, and variations of the expression are used interchangeably to refer to a biomarker value or level in a biological sample with a higher value or level (or range of values or levels) than those typically detected from biological sample similar to healthy or normal individuals. Multiple of the above terms may also refer to a biomarker value or level in a biological sample that is lower than the value or level (or range of values or levels) of a biomarker that may be detected in different steps of a particular disease.

Also, a biomarker that is highly expressed or poorly expressed may be referred to as an indication of normal progression or the absence of a disease or other condition in an individual, or has “differentially expressed” or a “differential level” or “differential value” compared to the “normal” expression level or value of the biomarker expressing the same. Thus, “differential expression” of a biomarker can also be manifested as a change in the “normal” expression level of the biomarker.

The terms “differential gene expression” and “differential expression” are used interchangeably to refer to the expression of an activated gene at a higher or lower level in a subject with a particular disease as compared to expression in a normal subject or a control subject. The term also encompasses the expression of an activated gene at high or low levels in mutually different steps of the same disease. Differential gene expression may comprise a comparison of expression between two or more genes or their gene products; or a comparison of expression ratios between two or more genes or their gene products; or, instead, a comparison of two products of the same gene that differ between a normal subject and a subject with the disease or between stages of the same disease, treated in different ways. Differential expression includes, for example, quantitative and qualitative differences in genes or their expression products between normal and diseased cells, or multiple cells undergoing mutually different disease events or disease stages, according to temporal or cellular expression patterns.

The present invention can determine gene expression using any method known in the art. It will be appreciated by those skilled in the art that the means for determining gene expression is not an important aspect of the present invention. In order to detect gene expression, a plurality of detection methods different from each other may be used, for example, a hybridization assay, a quality analysis, or a real-time fluorescent quantitative nucleic acid amplification assay. In certain embodiments, nucleic acid base sequence analysis methods can be used to detect gene sequences and detect biomarker values. An “increased” level with respect to a lncRNA gene product as referred to herein refers to a higher level than would normally be present. Typically, this can be estimated by comparison with a control. According to a particular embodiment, the increased level of lncRNA is a level that is 10%, 20%, 25%, 30%, 40%, 50%, 60%, 70%, 75%, 80%, 90%, 100%, 150%, 200% or even higher than the control. According to another particular embodiment, it is intended that the lncRNA gene product is expressed or present, whereas it is normally (or in a control) absent. In other words, in these embodiments, determining the increased expression of the lncRNA gene product corresponds to detecting the presence of the lncRNA gene product. Typically, in this case, controls will be included to ensure that the assay reaction is proceeding correctly. With respect to the “functional expression” of lncRNA, it means transcription and/or translation of a functional gene product. For non-protein encoding genes like lncRNA, “functional expression” can be deregulated at at least two levels. First, at the DNA level, for example, by deletion or disruption of the gene, or no transcription occurs (in both cases, synthesis of the relevant gene product is prevented). Deletions in transcription may result, for example, from epigenetic changes (e.g., DNA methylation) or from loss-of-function mutations.

Second, at the RNA level, e.g., by lack of efficient translation—e.g., because of instability of mRNA (e.g., by UTR variants), may result in degradation of mRNA prior to translation of the transcript. Or by lack of efficient transcription, e.g., because mutations induce new splice variants.

Accordingly, it is an object of the present invention to provide inhibitors of functional expression of the lncRNA gene. Such inhibitors may act at the DNA level or at the RNA (i.e., gene product) level. Since lncRNA is a non-coding gene, the gene has no protein product.

If inhibition is achieved at the DNA level, this can be done by knockout or disruption of the target gene using gene therapy. As used herein, a “knockout” may be a gene knockdown, or a gene may be knocked out by using techniques known in the art, including, but not limited to, retroviral gene transfer, causing a mutation, such as a point mutation, insertion, deletion, frame shift, or missense mutation. Another way in which a gene can be knocked out is by using zinc finger nucleases. Zinc finger nucleases (ZFN) are artificial restriction enzymes generated by fusing a zinc finger DNA binding domain with a DNA cleavage domain. Zinc finger domains can be engineered to target DNA sequences of interest, which allows zinc finger nucleases to target unique sequences in complex genomes. The reagents can be used to precisely alter the genome of higher organisms by exploiting endogenous DNA repair mechanisms. Other genome customization techniques that can be used to knockout genes are Meganuclease and TAL effector nucleases (TALENs, Cellectis bioresearch). It consists of a TALE DNA binding domain for sequence-specific recognition fused to the catalytic domain of an endonuclease that introduces a double strand DSB. Meganuclease is sequence-specific endonuclease, naturally occurring “DNA scissors”, derived from various unicellular organisms such as bacteria, yeast, algae, and certain plant organelles. Meganuclease has a long recognition site of 12 to 30 base pairs. The recognition site for the native Meganuclease can be altered to target the native genomic DNA sequence (e.g., an endogenous gene).

Another recent genome editing technique is the CRISPR/Cas system, which can be used to implement RNA-guided genome modification. CRISPR interference is a genetic technique that allows sequence-specific control of gene expression in prokaryotic and eukaryotic cells. It is based on the CRISPR (regularly clustered short palindromic repeat) pathway originating from the bacterial immune system.

Gene inactivation, i.e., suppression of functional expression of a gene, can also be achieved, for example, by designing transgenic organisms expressing antisense RNA, or by administering antisense RNA to a subject. The antisense construct can be delivered, for example, as an expression plasmid that, when expressed in a cell, produces an RNA that is complementary to at least one unique portion of a cellular lncRNA.

A faster method for suppressing gene expression is based on the use of shorter antisense oligomers consisting of DNA or other synthetic structural types such as phosphorothioate, 2′-O-alkylribonucleotide chimera, (locked nucleic acid) LNA, peptide nucleic acid (PNA) or morpholine nucleic acids. With the exception of RNA oligomers, PNA, and morpholine nucleic acids, all other antisense oligomers function in eukaryotic cells via RNA enzyme H-mediated target cleavage mechanisms. PNA and morpholine nucleic acids bind with high affinity and specificity to complementary DNA and RNA targets, thereby acting through simple steric hindrance to the RNA translation machinery, and appear to be completely resistant to nuclease attack. An “antisense oligomer” refers to an antisense molecule or anti-gene reagent comprising an oligomer of at least about 10 nucleotides in length. In embodiments, the antisense oligomer comprises at least 15, 18, 20, 25, 30, 35, 40, or 50 nucleotides. Antisense methods comprise designing oligonucleotides (DNA or RNA or derivatives thereof) complementary to the RNA encoded by the polynucleotide sequence of lncRNA. Antisense RNA can be introduced into a cell to inhibit the translation of a complementary mRNA by base pairing with it and physically blocking the translation machinery. The effect is therefore stoichiometric. Although perfect complementarity is preferred, this is not required. As referred to herein, a sequence is “complementary” to a portion of an RNA, meaning that the sequence has sufficient complementarity to hybridize with the RNA to form a stable duplex; in the case of double-stranded antisense polynucleotide sequences, single strands of the duplex DNA can be detected, or triplex formation can be detected. The ability to hybridize will depend on the degree of complementarity and the length of the antisense polynucleotide sequence. Generally, the longer the polynucleotide sequence undergoes hybridization, the more bases it may contain that mismatch with RNA and still form a stable duplex (or triplex, as the case may be). The skilled artisan can determine the tolerance for mismatches by measuring the melting point of the hybridization complex using standard procedures. The antisense oligomer should be at least 10 nucleotides in length, preferably the oligomer is 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules that have the same cleavage properties as enzymes and can be designed to target specific RNA sequences. Successful target gene inactivation, including time- and tissue-specific gene inactivation, using ribozymes has been reported in mice, zebrafish, and drosophila. RNA interference (RNAi) is a form of post-transcriptional gene silencing. The RNA interference phenomenon was first observed and described in Caenorhabditis elegans, where it was shown that exogenous double-stranded RNA (dsRNA) can specifically and vigorously disrupt the activity of genes containing homologous sequences through a mechanism that induces rapid degradation of the target RNA. Several reports describe the same catalytic phenomenon in other organisms, including experiments showing control of gene inactivation spatially and/or temporally, including plants (Arabidopsis), protozoa (Trypanosoma brucei), invertebrates (Drosophila melanogaster) and vertebrate species (zebrafish and Xenopus laevis). Mediated degradation of the sequence-specific messenger RNA may be a small interference RNA (siRNAs), generated from a longer dsRNA by ribonuclease III cleavage. Typically, siRNA is 20-25 nucleotides in length. siRNA typically comprises a sense RNA strand and a complementary antisense RNA strand annealed together by standard Watson Crick base pairing interactions (hereinafter “base pairing”). The sense strand comprises a nucleic acid sequence identical to the target sequence in the target mRNA. The sense and antisense strands of the siRNA of the invention may comprise two complementary single-stranded RNA molecules, or may comprise a single molecule in which the two complementary moieties base pair and are covalently linked by a single-stranded “hairpin” region (commonly referred to as shRNA). The term “isolated” means altered or removed from its natural state by human intervention. For example, a siRNA naturally occurring in a living animal is not “isolated”, but a synthetic siRNA or a siRNA partially or completely isolated from a material coexisting with its natural state is “isolated”. Isolated siRNA may exist in substantially pure form, or may exist in a non-native environment such as a cell into which siRNA is introduced.

The siRNA of the present invention can comprise partially purified RNA, substantially pure RNA, synthetic RNA, or recombinantly produced RNA, as well as altered RNA that differ from naturally occurring RNA by the addition, deletion, substitution, and/or alteration of one or more nucleotides. Such changes may include the addition of non-nucleotide material to, for example, the terminal (s) of the siRNA or to one or more internal nucleotides of the siRNA, including modifications that render the siRNA resistant to nuclease digestion.

One or both strands of the siRNA of the present invention may also comprise a 3′ overhang. “3′ overhang” refers to at least one unpaired nucleotide extending from the 3′ end of the RNA strand. Thus, in one embodiment, a siRNA of the invention comprises at least one 3′ overhang that is 1 to about 6 nucleotides in length (including ribonucleic acids or deoxyribonucleic acids), preferably 1 to about 5 nucleotides in length, more preferably 1 to about 4 nucleotides in length, and particularly preferably about 1 to about 4 nucleotides in length.

In embodiments where both strands of the siRNA molecule comprise a 3′ overhang, the length of the overhang may be the same or different for each strand. In a further embodiment, 3′ overhangs are present on both strands of siRNA, 2 nucleotides in length. To enhance the stability of the siRNA of the present invention, the 3′ overhangs may also be stabilized against degradation. In one embodiment, overhangs are stabilized by the inclusion of purine nucleotides such as adenosine or guanosine nucleotides.

Alternatively, the substitution of pyrimidine nucleotides with modified analogs, such as the substitution of uridine nucleotides in the 3′ overhang with 2′ deoxythymidine, is tolerated without affecting the efficiency of RNAi degradation. In particular, the deletion of the 2′ hydroxyl group in 2′ deoxythymidine significantly enhances nuclease resistance of the 3′ overhang in tissue culture media.

The siRNA of the present invention can be targeted to any segment of about 19 to 25 contiguous nucleotides in any target lncRNA RNA sequence (“target sequence”), examples of which are provided herein. Techniques for selecting target sequences for siRNA are well-known in the art. Thus, the sense strand of a siRNA of the invention can comprise a nucleotide sequence that is identical to about 19 to about 25 contiguous nucleotides of any stretch in the target mRNA.

The siRNA of the present invention can be obtained using a number of techniques known to those skilled in the art. For example, siRNA can be produced by chemical synthesis or recombinantly using methods known in the art. Preferably, the siRNA of the present invention is chemically synthesized using a suitably protected ribonucleoside phosphoramidite and a conventional DNA/RNA synthesizer. siRNA can be synthesized as two separate, complementary RNA molecules, or as a single RNA molecule with two complementary regions.

As used herein, an “effective amount” of siRNA is an amount sufficient to cause RNAi-mediated degradation of the target mRNA, or an amount sufficient to inhibit metastatic progression in a subject. RNAi-mediated degradation of a target mRNA can be detected by measuring the level of the target mRNA or protein in cells of the subject using standard techniques for isolating and quantifying mRNA or protein (as described above).

An effective amount of a siRNA of the present invention to be administered to a given subject can be readily determined by one of skill in the art by considering, for example, the size and weight of the subject, the extent of disease infiltration, the age, health, and sex of the subject, the route of administration, and whether the administration is local or systemic.

Another particular form of antisense RNA strategy is gapmer. Gapmer is chimeric antisense oligonucleotides comprising a central segment of deoxynucleotide monomers of sufficient length to induce RNase H cleavage. Flanking the central region of the Gapmer is a segment of 2′-O-modified ribonucleotides or other artificially modified ribonucleotide monomers, such as bridged nucleic acid (BNAs), which protects the inner segment from nuclease degradation. Gapmer has been used to achieve RNase-H mediated cleavage of the target RNA while reducing the number of phosphorothioate linkages. Phosphorothioates possess increased resistance to nucleases compared to unmodified DNA. However, they have several disadvantages. This comprises low binding capacity to complementary nucleic acids and non-specific binding to proteins leading to toxic side effects and limiting their use. The occurrence of toxic side effects and off-target effects caused by non-specific binding has prompted the design of new artificial nucleic acids for the development of modified oligonucleotides to provide effective and specific antisense activity in vivo without exhibiting toxic side effects. By recruiting RNaseH, gapmers selectively cleave the target oligonucleotide chain. Cleavage of this strand triggers an antisense effect. This method has proven to be a powerful method to suppress gene function and is becoming a popular method for antisense therapy. Gapmer is commercially available. Examples are LNA longRNA GapmeR supplied by Exiqon, or MOE gapmer supplied by Isis pharmaceuticals. MOE gapmers or “2′ MOE gapmers” are antisense phosphorothioate oligonucleotides of 15-30 nucleotides in which all backbone linkages are modified by the addition of a sulfur (phosphorothioate) on a non-bridging oxygen, and a stretch of at least 10 consecutive nucleotides remains unmodified (deoxy sugar), while the remaining nucleotides contain an O′-methyl O′-ethyl substitution at the 2′ position (MOE). For clinical use, the compounds according to the invention or prodrug forms thereof are formulated into pharmaceutical compositions that are formulated to be compatible with their intended route of administration, e.g., oral, rectal, parenteral, or other modes of administration. In general, pharmaceutical formulations are prepared by mixing the active substances with conventional pharmaceutically acceptable diluents or carriers. As used herein, the language “pharmaceutically acceptable carrier” is intended to comprise any solvents, dispersion media, coatings, antibacterial and antifungal reagents, absorption delaying reagents, and the like, compatible with pharmaceutical administration. Examples of pharmaceutically acceptable diluents or carriers are water, gelatin, acacia, lactose, microcrystalline cellulose, starch, sodium starch glycolate, calcium hydrogen phosphate, magnesium stearate, talc, colloidal silicon dioxide, and the like. The use of such media and reagents for pharmaceutically active substances is well-known in the art. Except insofar as any conventional media or reagent is incompatible with the active compound, use thereof in the compositions is contemplated.

The reagents of the invention may also be used in combination with other reagents for treating oral squamous cell carcinoma, and the other therapeutic compounds may be administered simultaneously with the main active ingredient, even in the same composition. Other therapeutic compounds may also be administered alone, in a separate composition, or in a dosage form different from the principal active ingredient. Partial dosages of the principal component may be administered concurrently with other therapeutic compounds, while other dosages may be administered separately. The dosage of the pharmaceutical composition of the present invention can be adjusted during the course of treatment depending on the severity of the symptoms, the frequency of recurrence, and the physiological response of the treatment regimen.

The term “sample” in the context of the present invention refers to a composition obtained from a target patient comprising cells and/or other molecular entities to be characterized and/or identified, e.g., according to physical, biochemical, chemical, and/or physiological characteristics. For example, the phrase “clinical sample” or “disease sample”, and variants thereof, refers to any sample obtained from a target patient in which it would be expected or known that cells and/or molecular entities, such as biomarkers, would be characterized.

The present invention will now be described in further detail with reference to the accompanying drawings and examples. The following examples are merely illustrative of the present invention and are not intended to limit the scope of the invention. Experimental procedures in which no specific conditions are specified in the examples generally follow conventional conditions, e.g., Sambrook et al. Molecular Cloning: Laboratory Manual (New York: Cold Spring Harbor Laboratory Press, 1989) or as recommended by the manufacturer.

### Example 1 Detection of Differential Expression of lncRNA by QPCR

1. Thirty-three cases of surrounding normal mucosa and oral squamous cell carcinoma tissues were collected and confirmed by pathological diagnosis, and all patients did not receive any form of treatment before surgery. The surgically excised samples were cryopreserved in liquid nitrogen.

2. RNA Extraction

The tissue sample cryopreserved in liquid nitrogen was taken out and placed in a pre-cooled mortar for grinding, and RNA was extracted and isolated according to the instructions in the kit. The details are as follows:

1) adding Trizol, and placing at room temperature for 5 min;

2) adding 0.2 ml chloroform, shaking the centrifuge tube vigorously, mixing well, and placing at room temperature for 5-10 min;

3) centrifuging at 12,000 rpm for 15 min, shifting the upper water phase into another new centrifuge tube (pay attention not to suck the protein material between two layers of water phase), adding an equal volume of isopropanol pre-cooled at −20° C., thoroughly reversing and mixing well, and placing on ice for 10 min;

4) after centrifuging at 12,000 rpm for 15 min at high speed, carefully discarding the supernatant, adding 75% DEPC ethanol in the proportion of 1 ml/ml Trizol to wash the precipitate (storing at 4° C.), washing the precipitate, shaking and mixing well, centrifuging at 12,000 rpm for 5 min at 4° C.;

5) discarding the ethanol liquid, placing at room temperature for 5 min, and adding DEPC water to dissolve the precipitate;

6) measuring the purity and concentration of RNA by Nanodrop2000 UV spectrophotometer and cryopreserved in a −70° C. refrigerator.

3. Reverse Transcription:

1) Preparing 10 μl Reaction System:

2 μl of MgCl2, 1 μl of 10×RT Buffer, 3.75 μl of water without Rnase, 1 μl of dNTP mixture, 0.25 μl of Rnase inhibitor, 0.5 μl of AMV reverse transcriptase, 0.5 μl of oligo dT aptamer primer and 1 μl of test sample were mixed.

2) Reverse Transcription Reaction Conditions

The reverse transcription reaction conditions in RNA PCR Kit (AMV) Ver. 3.0 were followed. 42° C. 60 min, 99° C. 2 min, 5° C. 5 min.

3) Polymerase Chain Reaction

1) Primer Design

QPCR amplification primers were designed based on the coding sequences of the RP11-875O11.3 gene and the GAPDH gene in Genebank and synthesized by Biomed co., Ltd. The specific primer sequences are shown in Table 1.

TABLE 1
Primer Sequences

| | Primer | Sequence |
|---|---|---|
| Gene | Sequence (5′-3′) | number |
| RP11- | CAGCCTCCAATTTCAGTA | SEQ ID NO: 1 |
| 875011.3 | CCCATCCCTCTCTTTATC | SEQ ID NO: 2 |
| LINC01679 | TGTCCTTCACTCCCATTT | SEQ ID NO: 3 |
| | GTAGCAAGAGCACTGTTC | SEQ ID NO: 4 |
| AP000695.4 | GCTAACATCATATCACAT | SEQ ID NO: 5 |
| | TTATCTGGAGAACTTCAA | SEQ ID NO: 6 |
| RP11- | AGGAGAGAATGGGAACTG | SEQ ID NO: 7 |
| 339B21.10 | GAGAACACAAACAAGGAATC | SEQ ID NO: 8 |
| RP11- | CAGAGGAAACGAAGACGATGTG | SEQ ID NO: 9 |
| 426C22.4 | AAGCCGCAGCCAATGAGA | SEQ ID NO: 10 |
| RP11- | TCCATCAACCAGACAATC | SEQ ID NO: 11 |
| 426C22.5 | ATTTACAAGTAGCCTCCAG | SEQ ID NO: 12 |
| AP000695.6 | AAGACAGCACATCCAGTA | SEQ ID NO: 13 |
| | TCAATCCTCCATCAATAACATT | SEQ ID NO: 14 |
| GAPDH | CTCTGGTAAAGTGGATATTGT | SEQ ID NO: 15 |
| | GGTGGAATCATATTGGAACA | SEQ ID NO: 16 |

2) Preparing 25 μl PCR Reaction System:

Forward (reverse) primer 1 μl, Takara Ex Taq HS 12.5 μl, template 2 μl, deionized water 8.5 μl

3) PCR reaction conditions: 94 C 4 min, (94° C. 20 s, 60° C. 30 s, 72° C. 30 s)×30 cycles.

Performing PCR reaction on the Light Cycler fluorescence quantitative PCR instrument using SYBR

Green as the fluorescent marker. The target bands were determined by melting curve analysis and electrophoresis. The relative quantification was performed by the 2-ΔΔCT method. Three replicates were performed for each sample. ΔΔCT method: ΔCT1=(target gene, sample to be detected) CT value-(internal reference gene, sample to be detected) CT value; ΔCT2=(target gene, control sample) CT value−(internal reference gene, control sample) CT value. ΔΔCT=ΔCT1-ΔCT2, expression multiple=2^{-ΔΔCT}.

5. Statistical Methods

GAPDH was used as an internal reference to calculate the fluorescence quantitative RT-PCR results of oral squamous cell carcinoma tissues and normal mucosal tissues. The difference between the two groups was statistically significant by t-test (P<0.05).

6. Results

Results are as shown in Table 2, compared with the surrounding normal mucosa tissues, RP11-875O11.3, LINC01679, AP000695.4, RP11-339B21.10, RP11-426C22.4, RP11-426C22.5, and AP000695.6 genes were up-regulated in oral squamous cell carcinoma tissues, the difference is statistically significant (P<0.05).

TABLE 2
Relative expression levels of lncRNA

| | Relative expression of genes (2^{-ΔΔCT}) | | |
|---|---|---|---|
| Gene | mean | SD | P Value |
| RP11-875O11.3 | 6.223 | 0.708 | 0.00606 |
| LINC01679 | 4.297 | 0.859 | 0.02188 |
| AP000695.4 | 12.867 | 1.105 | 0.00288 |
| RP11-339B21.10 | 20.930 | 1.781 | 0.00265 |
| RP11-426C22.4 | 13.033 | 1.315 | 0.00396 |
| RP11-426C22.5 | 32.093 | 2.205 | 0.00167 |
| AP000695.6 | 8.643 | 1.422 | 0.01134 |

### Example 2 Silence Detection and Functional Verification of lncRNA

1. Cell Culture

The human oral squamous cell carcinoma SCC-15 cells preserved in liquid nitrogen were recovered and inoculated in DMEM medium and cultured in an incubator at 37° C. and a constant temperature of 5% CO₂. After 24 h, the cells showed adherent growth, i.e., the fluid was changed once every 1-2 d after recovery, and trypsin was used to digest and prepare cell suspension for the experiment.

2. Cell Transfection

Cells were seeded at 2×10⁵/well into six-well cell culture plates and cultured in a 37° C., 5% CO₂ incubator. Cells in the logarithmic phase of proliferation (about 80%), after discarding the culture medium, were washed twice with PBS, 2 ml DMEM was added and starved for 1 h in an incubator, and transfection was performed using lipofectamine reagent 2000 (purchased from Invitrogen) according to the instructions. The experiments were divided into three groups: a blank control group (SCC-15), a negative control group (siRNA-NC), and an experimental group (siRNA group), wherein the negative control group siRNA has no homology with the sequence of each lncRNA gene.

Among them, siRNA-NC is a general negative control provided by Shanghai GenePharma Co., Ltd. The siRNA sequence for each lncRNA is shown in Table 3.

TABLE 3
siRNA sequences of IncRNA

| | | Sequence |
|---|---|---|
| Gene | siRNA | number |
| RP11- | UAUCAAAGUAGGAAUCAAGAA | SEQ ID NO: 17 |
| 875011.3 | CUUGAUUCCUACUUUGAUAAA | SEQ ID NO: 18 |
| LINC01679 | AAAUCCAAGGCAGUAGAAGCC | SEQ ID NO: 19 |
| | CUUCUACUGCCUUGGAUUUGC | SEQ ID NO: 20 |
| AP000695.4 | UUUGUUGAAAAAUAGCAUCUU | SEQ ID NO: 21 |
| | GAUGCUAUUUUUCAACAAAAU | SEQ ID NO: 22 |
| RP11- | UCUUUCUGCUCCUAAACACCU | SEQ ID NO: 23 |
| 339B21.10 | GUGUUUAGGAGCAGAAAGAAG | SEQ ID NO: 24 |
| RP11- | ACGUGAAUAUAGUACAUGCAC | SEQ ID NO: 25 |
| 426C22.4 | GCAUGUACUAUAUUCACGUGU | SEQ ID NO: 26 |
| RP11- | UUGCAAUUUGGCUUCAAUGCU | SEQ ID NO: 27 |
| 426C22.5 | CAUUGAAGCCAAAUUGCAAGC | SEQ ID NO: 28 |
| AP000695.6 | UUCUCAAUUCCAGAAUCGCCC | SEQ ID NO: 29 |
| | GCGAUUCUGGAAUUGAGAAGU | SEQ ID NO: 30 |

3. Detecting the Transcription Level of the RP11-875O11.3 Gene by QPCR

After 48 h of culture after transfection of each group of cells, total RNA was extracted using the Trizol method, reverse transcription, and real-time quantitative PCR detection were performed as in Example 1.

4. CCK-8 Cell Proliferation Assay

The cells in the negative control group and experimental group transfected for 24 h were digested by the conventional method, centrifuged, the supernatant was discarded, 1 ml complete medium was added to resuspend the cells, blown and mixed well, 3000 cells per well were inoculated into 96-well plate, complete medium was supplemented to 100 μl; 100 μl DEPC water was added to the outermost circle of the plate, and the 96-well plate was placed in a constant temperature incubator for culture. After incubation for 48 h, 100 μl medium containing 10% CCK-8 was added, continued to incubate in the incubator for 1 h, and then determine the absorbance at 450 nm on the microplate reader and got the statistical data.

5. Cell Migration Assay

The Transwell chamber was placed in a 24-well plate, 200 μl DMEM solution was added in the upper chamber, and placed in the incubator, hydrate for 1 h;

plating was performed according to 2×10⁴ cells in each chamber, the liquid in the upper chamber was supplemented to 200 μl, blown and mixed well, 700 μl complete medium was added in the lower chamber, and continued to culture in the incubator for 36 h; the chamber was taken out, the culture medium in the upper and lower chambers was discarded, the residual culture medium and cells in the upper chamber was gently wiped off with a cotton swab, the chamber was washed with PBS, shaken for 5 min, discard the PBS; 500 μl 4% paraformaldehyde was added into the lower chamber, fixed 30 min at room temperature, the fixing solution was discarded, washed with PBS for 3 times, shaked for 5 min, the PBS was discarded; the chamber was placed in a fume hood and air dried for 30 min; 500 μl of prepared 0.1% crystal violet solution was added into the lower chamber, air bubbles were removed, and standing for 30 min; the crystal violet solution was discarded, PBS was used to clean it for 3 times, shaking for 5 min on a shaker, discarding PBS, the excess liquid in the upper chamber was gently wiped off with a dry cotton swab, the chamber was placed under a microscope, and cell counting was performed.

6. Statistical Methods

The experiments were performed in triplicate, and the results were expressed as mean±standard deviation. The t-test was used for the difference between the two, which was considered statistically significant when P<0.05.

7. Results

The silencing effect of siRNA was shown in Table 4. Compared with the blank control group, each siRNA in the experimental group had a better interference effect on the corresponding gene (P<0.05), while siRNA-NC had no significant change (P>0.05).

TABLE 4
Transfection effect of siRNA

| | Relative expression | | | Relative expression | | |
|---|---|---|---|---|---|---|
| | of genes in the | | | of genes in | | |
| | siRNA-NC group (2-^{ΔΔCT}) | | | siRNA group (2-^{ΔΔCT}) | | |
| Gene | mean | SD | P Value | mean | SD | P Value |
| RP11-875O11.3 | 0.967 | 0.021 | 0.10913 | 0.283 | 0.042 | 0.00112 |
| LINC01679 | 0.983 | 0.023 | 0.33773 | 0.373 | 0.025 | 0.00054 |
| AP000695.4 | 0.963 | 0.015 | 0.05327 | 0.160 | 0.046 | 0.00099 |
| RP11-339B21.10 | 0.940 | 0.036 | 0.10224 | 0.103 | 0.035 | 0.00051 |
| RP11-426C22.4 | 0.970 | 0.026 | 0.18850 | 0.210 | 0.050 | 0.00133 |
| RP11-426C22.5 | 0.980 | 0.017 | 0.18350 | 0.127 | 0.047 | 0.00097 |
| AP000695.6 | 0.960 | 0.020 | 0.07418 | 0.177 | 0.040 | 0.00080 |

Note:
P: compared with blank control

CCK-8 test results are shown in Table 5, the OD value of the experimental group was significantly lower than that of the negative control group, P<0.05, indicating that the lncRNA in this study plays an important role in the proliferation of oral squamous carcinoma cells.

TABLE 5
OD values

OD value

| | | | |
|---|---|---|---|
| Grouping | Mean | SD | P value |

| | | | | |
|---|---|---|---|---|
| Negative | siRNA-NC | 1.200 | 0.056 | — |
| control group | | | | |
| Experimental | RP11-875O11.3 | 0.677 | 0.021 | 0.00124 |
| group | LINC01679 | 0.540 | 0.050 | 0.00529 |
| | AP000695.4 | 0.730 | 0.040 | 0.01189 |
| | RP11-339B21.10 | 0.883 | 0.032 | 0.02112 |
| | RP11-426C22.4 | 0.773 | 0.045 | 0.01325 |
| | RP11-426C22.5 | 0.627 | 0.025 | 0.00243 |
| | AP000695.6 | 0.830 | 0.020 | 0.00881 |

Results of the migration assay are shown in Table 6, compared with the negative control group, the number of migrated cells in the experimental groups RP11-875O11.3, LINC01679, AP000695.4, RP11-339B21.10, and AP000695.6 was significantly decreased (P<0.05), while the number of migrated cells in the RP11-426C22.4 and RP11-426C22.5 groups was decreased, but not significantly. According to the above results, RP11-875O11.3, LINC01679, AP000695.4, RP11-339B21.10, and AP000695.6 played an important role in the metastasis of oral squamous cell carcinoma.

TABLE 6
Number of migrated cells

| | | Number of | | |
|---|---|---|---|---|
| | | transmembrane | | |
| | | cells | | |
| Grouping | | Mean | SD | P value |
| Negative | siRNA-NC | 109.7 | 11.015 | — |
| control group | | | | |
| Experimental | RP11-875O11.3 | 65.3 | 4.041 | 0.02730 |
| group | LINC01679 | 53.0 | 2.000 | 0.00834 |
| | AP000695.4 | 72.0 | 3.606 | 0.04234 |
| | RP11-339B21.10 | 77.3 | 3.055 | 0.04446 |
| | RP11-426C22.4 | 81.7 | 6.110 | 0.10496 |
| | RP11-426C22.5 | 89.0 | 3.000 | 0.12511 |
| | AP000695.6 | 73.7 | 2.517 | 0.04399 |

The above description of the embodiments is intended only to understand the method of the invention and the core idea thereof. It will be appreciated by those skilled in the art that numerous modifications and variations can be made to the present invention without departing from the principles of the invention. Such modifications and variations are intended to be within the scope of the appended claims.

1-13. (canceled)

14. A biomarker for diagnosing oral squamous cell carcinoma, characterized in that the biomarker is selected from one or more of RP11-875O11.3, LINC01679, AP000695.4, RP11-339B21.10, RP11-426C22.4, RP11-426C22.5, AP000695.66.

15. The biomarker of claim 14, characterized in that the biomarker is significantly up-regulated in oral squamous cell carcinoma.

16. A method of screening a candidate drug for preventing or treating oral squamous cell carcinoma, characterized in that the method comprises:
(1) treating a system expressing or containing the biomarker of claim 14 with a substance to be screened; and
(2) detecting the expression level of the biomarker in the system;
if the substance to be screened reduces the expression level of the biomarker, indicating that the substance to be screened is a candidate drug for preventing or treating oral squamous cell carcinoma.

17. A method for inhibiting the proliferation of a tumor cell, characterized in that the method comprises introducing into the tumor cell an inhibitor of the biomarker of claim 14.

18. A method for diagnosing oral squamous cell carcinoma, characterized by comprising: detecting the expression level of the biomarker of claim 14 in a sample from a subject; if the expression of at least one of RP11-875O11.3, LINC01679, AP000695.4, RP11-339B21.10, RP11-426C22.4, RP11-426C22.5 and AP000695.6 in the sample of the subject is significantly increased as compared with that in normal human, the subject is diagnosed as an oral squamous cell carcinoma patient.

19. The method of claim 18, characterized in that reagents for detecting the biomarker are selected from: primers that specifically amplify RP11-875O11.3, LINC01679, AP000695.4, RP11-339B21.10, RP11-426C22.4, RP11-426C22.5 and/or AP000695.6 genes; or probes that specifically recognize RP11-875O11.3, LINC01679, AP000695.4, RP11-339B21.10, RP11-426C22.4, RP11-426C22.5 and/or AP000695.6 genes.

20. The method of claim 19, characterized in that the primer sequences for specifically amplifying RP11-875O11.3, LINC01679, AP000695.4, RP11-339B21.10, RP11-426C22.4, RP11-426C22.5 and/or AP000695.6 genes are shown in SEQ ID Nos: 1-14, respectively.

21. The method of claim 18, characterized in that the method for detecting the biomarkers comprises reverse transcription PCR, real-time quantitative PCR, in situ hybridization, or gene chip.

22. The method of claim 21, characterized in that reagents for detecting the biomarker gene by reverse transcription PCR comprise at least one pair of primers for specifically amplifying the biomarker; reagents for detecting the biomarker by real-time quantitative PCR comprise at least one pair of primers for specifically amplifying the biomarker; reagents for detecting the biomarker by in situ hybridization comprise probes that hybridize to nucleic acid sequences of the biomarker; reagents for detecting the biomarker by gene chip comprise probes that hybridize to nucleic acid sequences of the biomarkers.

23. The method of claim 19, characterized in that products for diagnosing oral squamous cell carcinoma comprise a chip, a kit, or a test strip.

24. The method of claim 18, characterized in that the method comprises:
(1) collecting subject samples;
(2) extracting RNA from the sample from the subject and detecting the expression level of the biomarker;
(3) if the expression of at least one of RP11-875O11.3, LINC01679, AP000695.4, RP11-339B21.10, RP11-426C22.4, RP11-426C22.5 and AP000695.6 in the sample of the subject is significantly increased as compared with that in normal human, the subject is diagnosed as an oral squamous cell carcinoma patient.

25. A method for preventing or treating oral squamous cell carcinoma, characterized in that the method comprises: administering to a subject a pharmaceutically effective amount of an inhibitor of the biomarker of claim 14.

26. The method of claim 25, characterized in that the inhibitor reduces the expression level of the one or more biomarkers.

27. The method of claim 26, characterized in that the inhibitor is selected from the group consisting of gapmer, interference RNA, CRISPR, TALEN, or zinc finger nucleases.

28. The method of claim 27, characterized in that the inhibitor is selected from interference RNA.

29. The method of claim 28, the sequence of the interference RNA is selected from the group consisting of SEQ ID Nos: 17-30.

30. A method for preventing or treating oral squamous cell carcinoma, characterized in that the method comprises: administering to a subject a pharmaceutical composition, the pharmaceutical composition comprises an inhibitor of functional expression of the biomarker of claim 14.

31. The method of claim 30, characterized in that the inhibitor reduces the expression level of the one or more biomarkers.

32. The method of claim 31, characterized in that the inhibitor is selected from the group consisting of gapmer, interference RNA, CRISPR, TALEN, or zinc finger nucleases.

33. The method of claim 30, characterized in that the pharmaceutical composition further comprises a pharmaceutically acceptable carrier.


SEQUENCE LISTING (30 sequences)

SEQ ID NO: 1 - DNA, 18 bp, Artificial Sequence
  synthesized
cagcctccaatttcagta

SEQ ID NO: 2 - DNA, 18 bp, Artificial Sequence
  synthesized
cccatccctctctttatc

SEQ ID NO: 3 - DNA, 18 bp, Artificial Sequence
  synthesized
tgtccttcactcccattt

SEQ ID NO: 4 - DNA, 18 bp, Artificial Sequence
  synthesized
gtagcaagagcactgttc

SEQ ID NO: 5 - DNA, 18 bp, Artificial Sequence
  synthesized
gctaacatcatatcacat

SEQ ID NO: 6 - DNA, 18 bp, Artificial Sequence
  synthesized
ttatctggagaacttcaa

SEQ ID NO: 7 - DNA, 18 bp, Artificial Sequence
  synthesized
aggagagaatgggaactg

SEQ ID NO: 8 - DNA, 20 bp, Artificial Sequence
  synthesized
gagaacacaaacaaggaatc

SEQ ID NO: 9 - DNA, 22 bp, Artificial Sequence
  synthesized
cagaggaaacgaagacgatgtg

SEQ ID NO: 10 - DNA, 18 bp, Artificial Sequence
  synthesized
aagccgcagccaatgaga

SEQ ID NO: 11 - DNA, 18 bp, Artificial Sequence
  synthesized
tccatcaaccagacaatc

SEQ ID NO: 12 - DNA, 19 bp, Artificial Sequence
  synthesized
atttacaagtagcctccag

SEQ ID NO: 13 - DNA, 18 bp, Artificial Sequence
  synthesized
aagacagcacatccagta

SEQ ID NO: 14 - DNA, 22 bp, Artificial Sequence
  synthesized
tcaatcctccatcaataacatt

SEQ ID NO: 15 - DNA, 21 bp, Artificial Sequence
  synthesized
ctctggtaaagtggatattgt

SEQ ID NO: 16 - DNA, 20 bp, Artificial Sequence
  synthesized
ggtggaatcatattggaaca

SEQ ID NO: 17 - RNA, 21 nt, Artificial Sequence
  synthesized
uaucaaaguaggaaucaagaa

SEQ ID NO: 18 - RNA, 21 nt, Artificial Sequence
  synthesized
cuugauuccuacuuugauaaa

SEQ ID NO: 19 - RNA, 21 nt, Artificial Sequence
  synthesized
aaauccaaggcaguagaagcc

SEQ ID NO: 20 - RNA, 21 nt, Artificial Sequence
  synthesized
cuucuacugccuuggauuugc

SEQ ID NO: 21 - RNA, 21 nt, Artificial Sequence
  synthesized
uuuguugaaaaauagcaucuu

SEQ ID NO: 22 - RNA, 21 nt, Artificial Sequence
  synthesized
gaugcuauuuuucaacaaaau

SEQ ID NO: 23 - RNA, 21 nt, Artificial Sequence
  synthesized
ucuuucugcuccuaaacaccu

SEQ ID NO: 24 - RNA, 21 nt, Artificial Sequence
  synthesized
guguuuaggagcagaaagaag

SEQ ID NO: 25 - RNA, 21 nt, Artificial Sequence
  synthesized
acgugaauauaguacaugcac

SEQ ID NO: 26 - RNA, 21 nt, Artificial Sequence
  synthesized
gcauguacuauauucacgugu

SEQ ID NO: 27 - RNA, 21 nt, Artificial Sequence
  synthesized
uugcaauuuggcuucaaugcu

SEQ ID NO: 28 - RNA, 21 nt, Artificial Sequence
  synthesized
cauugaagccaaauugcaagc

SEQ ID NO: 29 - RNA, 21 nt, Artificial Sequence
  synthesized
uucucaauuccagaaucgccc

SEQ ID NO: 30 - RNA, 21 nt, Artificial Sequence
  synthesized
gcgauucuggaauugagaagu